(19) United States
(12) Patent Application Publication
(10) Pub. No.: US 2017/0265785 A1
(43) Pub. Date: Sep. 21, 2017

(54) AUTOMATIC CLASSIFICATION AND USE OF USER MOVEMENTS

(71) Applicant: Intel Corporation, Santa Clara, CA (US)

(72) Inventors: Robert L. Vaughn, Portland, OR (US); Jeffrey C. Sedayao, San Jose, CA (US); Casey L. Baron, Chandler, AZ (US)

(21) Appl. No.: 15/076,006

(22) Filed: Mar. 21, 2016

(51) Int. Cl. A61B 5/11; A61B 8/00; A61B 5/16; A61B 5/00

(52) U.S. Cl. CPC A61B 5/1123; A61B 5/7267; A61B 5/6801; A61B 5/6887; A61B 8/00; A61B 5/1113; A61B 5/1112; A61B 5/1128; A61B 5/0022; A61B 5/742; A61B 5/16; A61B 2562/0219

(57) ABSTRACT

Apparatus and method to facilitate motion-related diagnosis or monitoring are disclosed herein. First sensor based data and second sensor based data associated with a user for a first and second time period may be received, from a first device. When the first and second sensor based data are determined to be associated with the first and second motion patterns, identifying the first and second sensor based data to be first and second motions by the user. In response to receiving, from a second device, a motion-related query associated with the user, searching among the first motion and the second motion to determine a query match. And providing the query match comprising data associated with the first motion or the second motion.

### TECHNICAL FIELD

The present disclosure relates generally to the technical field of computing, and more particularly, to computing systems for capturing and/or using data associated with user movement.

### BACKGROUND

The background description provided herein is for the purpose of generally presenting the context of the disclosure. Unless otherwise indicated herein, the materials described in this section are not prior art to the claims in this application and are not admitted to be prior art or suggestions of the prior art, by inclusion in this section.

A person's movements throughout the day and night, or over multiple time periods, such as days, weeks, or months, may be captured using dedicated devices, typically in particular physical contact with the person. Data captured by the dedicated devices may be stored for later use. Because the data may comprise a large volume of sensor data, the data may not be readily understandable or useful for the person or interested parties. For example, data from a personal “flight recorder” or “black box” may comprise a large amount of data, but the data, in of itself, is of limited value. Similarly, in order to track objects associated with a person (e.g., keys), such objects may be equipped with dedicated tagging equipment, such as radio frequency identification (RFID) tags. However, adding dedicated tagging equipment to many or all of a person's objects are impractical.

### BRIEF DESCRIPTION OF THE DRAWINGS

Embodiments will be readily understood by the following detailed description in conjunction with the accompanying drawings. The concepts described herein are illustrated by way of example and not by way of limitation in the accompanying figures. For simplicity and clarity of illustration, elements illustrated in the figures are not necessarily drawn to scale. Where considered appropriate, like reference labels designate corresponding or analogous elements.

FIG. 1 depicts a block diagram of an example system for practicing the present disclosure, according to some embodiments.

FIG. 2 depicts a block diagram illustrating details of the system of FIG. 1, according to some embodiments.

FIG. 3 depicts an example process for training or building a library of motion patterns, according to some embodiments.

FIGS. 4A-4D depict graphs illustrating example sensor based data associated with particular motions, according to some embodiments.

FIG. 5 depicts an example process for automatically determining or classifying user movements, according to some embodiments.

FIG. 6 depicts an example process for using the user motions determined using the process of FIG. 5, according to some embodiments.

FIG. 7 depicts an example computing environment suitable for practicing various aspects of the present disclosure, according to some embodiments.

FIG. 8 depicts an example non-transitory computer-readable storage medium having instructions configure to practice all or selected ones of the operations associated with the processes described in reference to FIG. 1-6.

### DETAILED DESCRIPTION

Computing apparatuses, methods and storage media for facilitating motion-related diagnosis or monitoring are described herein. In some embodiments, an apparatus may include one or more processors; one or more storage medium to store a plurality of motion patterns; a motion analysis module; and a query module. The motion analysis module having first instructions to be executed by the one or more processors, to determine and store motion records for a user, wherein the motion analysis module is to receive, from a first device, first and second sensor data of the user at a first and a second time period, determine and store first and second motion records for the user based at least in part on the first and second sensor data, and the plurality of motion patterns. The query module having second instructions to be executed by the one or more processors, to provide motion-related query match, wherein the query module is to receive, from a second device, a motion-related query associated with the user, search among the first and second motion records to determine a query match; and provide, to the second device, the query match including data associated with the first motion record or the second motion record. These and other aspects of the present disclosure will be more fully described below.

In the following detailed description, reference is made to the accompanying drawings which form a part hereof wherein like numerals designate like parts throughout, and in which is shown by way of illustration embodiments that may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. Additionally, it should be appreciated that items included in a list in the form of “at least one A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

The disclosed embodiments may be implemented, in some cases, in hardware, firmware, software, or any combination thereof. The disclosed embodiments may also be implemented as instructions carried by or stored on one or more transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device). As used herein, the term “logic” and “module” may refer to, be part of, or include an application specific integrated circuit (ASIC), an electronic circuit, a processor (shared, dedicated, or group), and/or memory (shared, dedicated, or group) that execute one or more software or firmware programs, a combinational logic circuit, and/or other suitable components that provide the described functionality.

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, it may not be included or may be combined with other features.

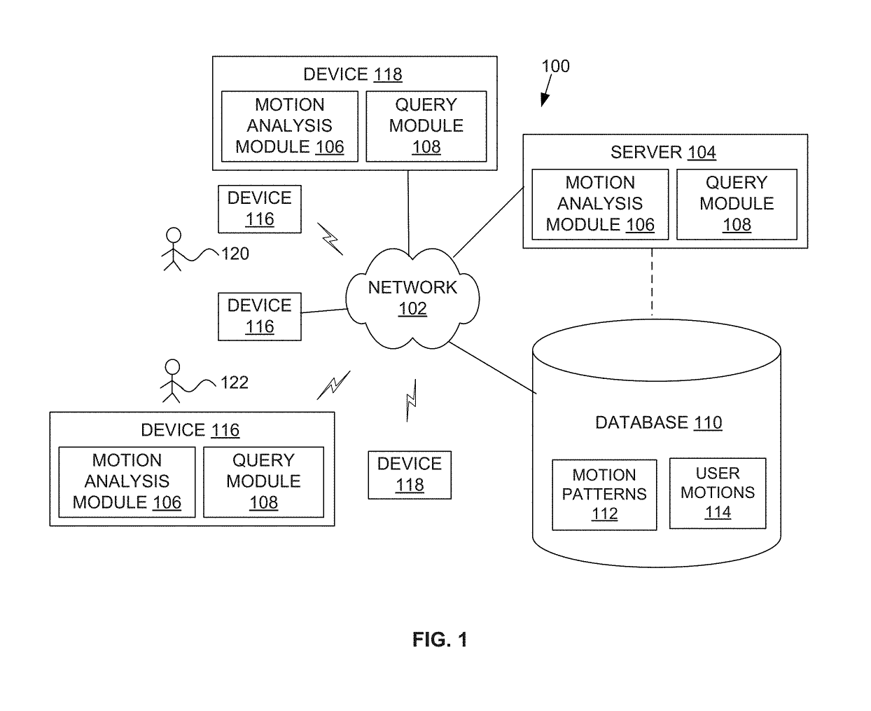

FIG. 1 depicts a block diagram of an example system 100 for practicing the present disclosure, according to some embodiments. System 100 may include a network 102, a server 104, a database 110, devices 116, and devices 118. Each of the server 104, database 110, devices 116, and devices 118 may communicate with the network 102.

Network 102 may comprise a wired and/or wireless communications network. Network 102 may include one or more network elements (not shown) to physically and/or logically connect computing devices to exchange data with each other. In some embodiments, network 102 may be the Internet, a wide area network (WAN), a personal area network (PAN), a local area network (LAN), a campus area network (CAN), a metropolitan area network (MAN), a virtual local area network (VLAN), a cellular network, a WiFi network, a WiMax network, and/or the like. Additionally, in some embodiments, network 102 may be a private, public, and/or secure network, which may be used by a single entity (e.g., a business, school, government agency, household, person, and the like). Although not shown, network 102 may include, without limitation, servers, databases, switches, routers, base stations, repeaters, software, firmware, intermediating servers, and/or other components to facilitate communication.

Server 104 may comprise one or more computers, processors, or servers to perform the motion analysis and query functionalities described herein. In some embodiments, server 104 may communicate with database 110 (directly or indirectly via network 102), devices 116, and/or devices 118 via network 102. Server 104 may host one or more applications accessed by the devices 116 and/or 118; provide processing functionalities for the devices 116 and/or 118; provide data to the devices 116 and/or 118; perform motion analysis, determination, and/or classification functionalities; perform searches to identify matching (or best matching) query results to motion-related queries; facilitates access to and/or store information in the database 110; and the like. In some embodiments, server 104 may include one or more web servers, one or more application servers, one or more servers providing user interface (UI) or graphical user interface (GUI) functionalities in connection with populating and/or accessing database 110, and the like.

Database 110 may comprise one or more storage devices to store data and/or instructions for use by devices 116, devices 118, and/or server 104. The content of database 110 may be accessed via network 102 and/or directly by the server 104. The content of database 110 may be arranged in a structured format to facilitate selective retrieval. In some embodiments, the content of database 110 may include, without limitation, a plurality of motion patterns 112 (also referred to as motion signatures, motion patterns library, or motion signatures library), a plurality of user motions 114 derived or determined using sensor based data and the plurality of motion patterns 112, and the like. In some embodiments, database 110 may comprise more than one database, a first database including the plurality of motion patterns 112 and a second database including the plurality of user motions 114.

Devices 116 may comprise wired and/or wireless communication computing devices in communication with network 102. Devices 116 may comprise laptops, computers, work stations, smart phones, tablets, Internet of Things (IoT) devices, wearable devices, set top boxes, appliances, vehicles, cameras, microphones, image capture devices, audio capture devices, geographical location sensing devices, or any other types of devices that include at least a component (e.g., sensor) capable of capturing information about one or more movements made by a first user 120 and/or a second user 122. One or more of the devices 116 may be used to capture movement information about a particular user at a particular point in time. Devices 116 may be in physical contact or not in physical contact with the first user 120 and/or second user 122 during capture of the movement information. In some embodiments, devices 116 may communicate with database 110, server 104, and/or devices 118 via network 102. Devices 116 may perform motion analysis, determination, and/or classification functionalities; perform searches to identify matching (or best matching) query results to motion-related queries; facilitates access to and/or store information in the database 110; and the like. Devices 116 may be geographically distributed from each other and/or the network 102. Although three devices 116 are shown in FIG. 1, more or less than three devices may be included in the system 100.

Devices 118 may comprise wired and/or wireless communication computing devices in communication with network 102. In some embodiments, devices 118 may include, without limitation, one or more input mechanisms (e.g., keyboard, trackball, trackpad, touch screen, mouse, etc.), displays (e.g., touch screens), processors, storage unit, and transceivers to receive queries from users and to present query results to the users. In some embodiments, devices 118 may be similar to devices 116. In some embodiments, devices 118 may communicate with database 110, server 104, and/or devices 116 via network 102. Devices 118 may perform motion analysis, determination, and/or classification functionalities; perform searches to identify matching (or best matching) query results to motion-related queries; facilitates access to and/or store information in the database 110; and the like. Devices 118 may be geographically distributed from each other and/or the network 102. Although two devices 118 are shown in FIG. 1, more or less than two devices may be included in the system 100.

In some embodiments, a device that captures user movement information may be different from a device that recalls or uses the user movement information, such as via a motion-related query. For example, device 116 may comprise a movement capture device while a device 118 comprises a querying device. In other embodiments, the same device (e.g., device 116 or 118) may capture user movement information and also recall/use the user movement information, such as via a motion-related query.

In some embodiments, server 104, devices 116, and/or devices 118 may include one or both of a motion analysis module 106 and a query module 108. As described in detail below, the motion analysis module 106 may be configured to facilitate determination, generation, and/or access to the plurality of motion patterns 112 and the plurality of user motions 114 in database 110. Query module 108 may be responsive to one or more motion-related queries for particular user motions from among the plurality of user motions 114. Depending on computing and user experience requirements such as, but not limited to, processing capabilities, communication bandwidth and speed, user experience expectations, computing architecture, application use and license model, data security, and the like, the functionalities of one, both, or part of one or both of the motion analysis module 106 and query module 108 may be performed by one of the server 104, devices 116, or devices 118. For example, if a large volume of data is analyzed, server 104 may be better suited to perform such functions than devices 116 or 118. As another example, if the devices 116 and 118 are considered to be “slaves” in a master-slave architecture, server 104 may perform the processing functions and provide the processed results (e.g., query results) to the devices 116, 118. As still another example, because the user motions 114 include users' sensor based data classified into specific motions, querying the user motions 114 may be less processing intensive and thus, the query module 108 may be included in the devices 116, 118.

Although a single server 104 and database 110 are shown in FIG. 1, each of server 104 and database 110 may comprise two or more components and/or may be located at one or more geographically distributed location from each other. Alternatively, database 110 may be included within server 104. Furthermore, while system 100 shown in FIG. 1 employs a client-server architecture, embodiments of the present disclosure are not limited to such an architecture, and may equally well find application in, for example, a distributed or peer-to-peer architecture system.

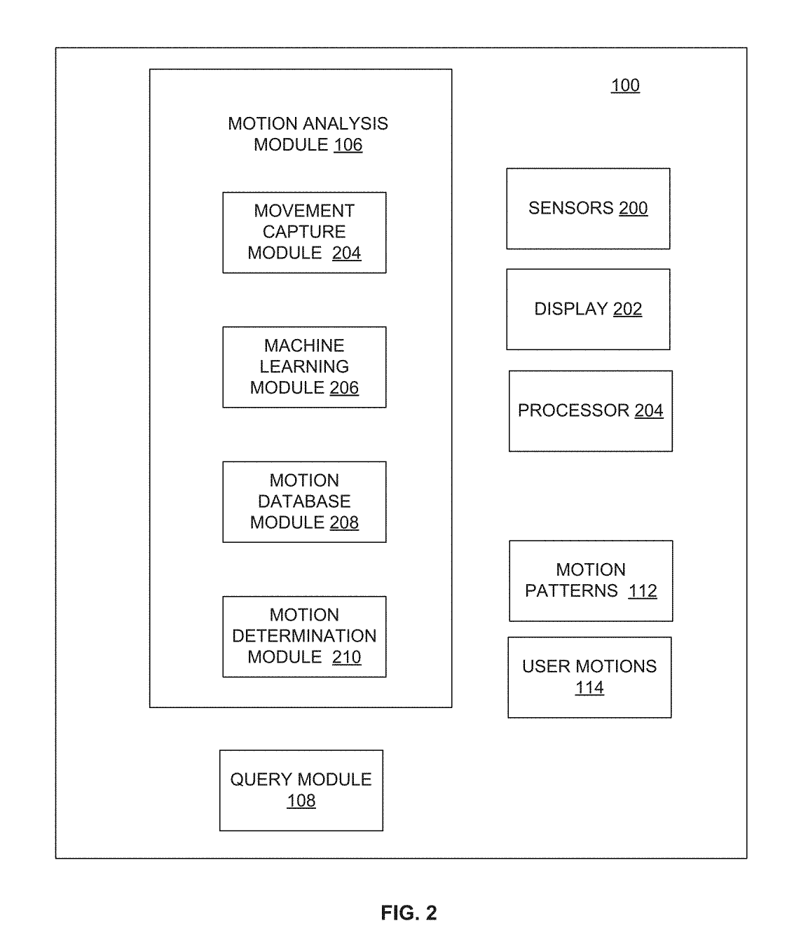

FIG. 2 depicts a block diagram illustrating details of the system 100, according to some embodiments. The system 100 may include sensors 200, a display 202, a processor 204, the motion analysis module 106, the query module 108, the motion patterns 112, and the user motions 114. In some embodiments, sensors 200 may be integrated, attached, or coupled to one or more of devices 116 and/or 118. Sensors 200 may be capable of capturing information about one or more movements made by the first and/or second users 120, 122. Sensors 200 may comprise, without limitation, an accelerometer, a gyroscope, a barometric sensor, an ultrasonic sensor, a motion sensor, a location sensor, a global positioning system (GPS), an audio sensor, a visual sensor, a camera, an infrared sensor (e.g., passive infrared (PIR) sensor), radio detection and ranging (RADAR), a light radar (LIDAR), a tomographic sensor, a vibration sensor, and the like.

Display 202 may be integrated, attached, or coupled to one or more of devices 116 and/or 118. Display 202 may be capable of displaying an interface to receive motion-related queries or requests and provide query matches corresponding to the motion-related queries, wherein the query matches may comprise one or more particular motions and/or associated information relating to the user specified in a query, from the user motions 114. Details about the user motions 114 are provided in the sections below.

Processor 204 may comprise one or more processors that are included in server 104, database 110, devices 116, and/or devices 118. In some embodiments, processor 204 may be capable of controlling the sensors 200 and/or display 202; executing instructions to perform one or more of the functionalities disclosed herein; and/or the like. For example, processor 204 may execute instructions embodied in the motion analysis module 106 and/or the query module 108. As another example, processor 204 may execute instructions to create, access, and/or maintain data in the database 110 such as motion patterns 112 and user motions 114.

Motion analysis module 106 (also referred to as a motion analysis engine) may include a movement capture module 204, a machine learning module 206, a motion database module 208, and a motion determination module 210. Query module 108 may also be referred to as a query engine or selective motion recall engine. Motion analysis module 106, movement capture module 204, a machine learning module 206, a motion database module 208, motion determination module 210, and query module 108 may comprise one or more software components, programs, applications, apps, or other units of code base or instructions configured to be executed by the processor 204. Modules 106 and 108 may communicate with each other and access the motion patterns 112 and user motions 114. Although modules 106, 108, and 204-210 are shown as distinct modules in FIG. 2, these modules may be implemented as fewer or more modules than illustrated. Any of modules 106, 108, and 204-210 may also communicate with one or more components included in the system 100.

Motion capture module 204 may receive sensor based data associated with movement of a user's body or body part(s) (e.g., first user 120, second user 122, etc.). Body parts may include, without limitation, hands, feet, toes, fingers, legs, torso, back, upper body, lower body, head, neck, part of the arm, part of the leg, and any other possible part of the body. Sensor based data may be generated by devices 116, 118 that captured movement information associated with the user using one or more sensors. Sensor based data, in some embodiments, may comprise raw sensor data outputted from the one or more sensors, or sensor derived data, which may be raw sensor data that have been processed (e.g., filtered, normalized, weighted, converted, transformed, compressed, encrypted, or otherwise refined from the raw form) before sending to the motion capture module 204. Motion capture module 204 may process the received sensor based data, or further process the received sensor based data when the sensor based data comprises processed data, suitable for use by the machine learning module 206 and/or motion determination module 210.

During a training or library building phase, sets of sensor based data associated with respective known movements by one or more users may be analyzed by the machine learning module 206. As described in detail below, machine learning module 206 may perform statistical, cluster, and other analyses to determine what sensor based data (and any variations, ranges, and other associated parameters) defines a particular movement, which in turn, permits the particular movement to be classified or typed as a particular motion. Each of the defining set of sensor based data associated with a particular motion may be referred to as a motion pattern. In some embodiments, a motion pattern may be associated with a plurality of users, a particular group of users, or a particular user. For example, the sensor based data associated with sipping coffee is likely to differ from sensor based data associated with throwing a ball. As another example, sensor based data associated with throwing a ball by professional baseball pitchers may differ from throwing a ball by non-baseball professionals. In some embodiments, as additional sets of sensor based data become available over time, the machine learning module 206 may perform additional analysis to refine and/or update the motion patterns, as appropriate.

These motion patterns determined or derived by the machine learning module 206 may then be stored in the database 110 by the motion database module 208. Motion database module 208 may organize, annotate, tag, and/or otherwise format each of the motion patterns for storage and subsequent use. For example, each of the motion patterns 112 may include, without limitation, a motion identifier; a defining set of sensor based data for the particular motion; any variations, ranges, or other parameters relating to determining what sensor based data corresponds to the particular motion; metadata tags; and the like. Motion database module 208 may also facilitate retrieval of select motion patterns 112 to determine user motions during a user motion determination phase.

During the user motion determination or identification phase, movement capture module 204 may receive sensor based data associated with movement by a particular user (e.g., first user 120) during a particular time period. In contrast to the training or library building phase, the movement is not known or pre-determined by the system 100. Hence, motion determination module 210 may use the motion patterns 112 to determine what motion, from among the plurality of motions defined by the motion patterns 112, corresponds to the received sensor based data.

Once the received sensor based data has been classified or typed based on the motion patterns 112, the determined motion may be stored in the database 110 as part of the user motions 114. Each of the user motions 114 may comprise a record of at least a particular movement made by a particular user during a particular time period that has been identified as a particular motion. In some embodiments, each of the user motions 114 may comprise a record including, but not limited to, a user identifier, a motion identifier, location information, a date and time stamp, spatial orientation, metadata tags, and/or motion characteristics such as speed and range (which are within defined parameters but still unique for the identified movement). User motions 114 may include records for one or more users, more than one record for a particular user, and the like.

The query module 108 may receive motion-related queries provided to the devices (e.g., devices 116, 118) by users (e.g., first and second users 120, 122), and in response, determine and provide query results that best match respective motion-related queries to the respective querying devices. Query module 108 may be configured to search for one or more motions from among the user motions 114. Query results may comprise identification of particular motion(s) (and/or related information) associated with a particular user. A query may be made by the same user about whom motion information is sought. For instance, the first user 120 may have lost his keys and composes a query to recall his movements around a particular time period when the keys were likely misplaced. The query result may comprise the motions and/or locations associated with the first user 120 during the particular time period stored in the user motions 114. Alternatively, a query may be made by a different user than the user about whom motion information is sought. For example, the motion information may be based on movements made by the first user 120 while the query is made by the second user 122 and, optionally, on a device different from the device(s) that captured the movements made by the first user 120. The second user 122 may be, for example, the first user's 120 doctor searching for particular motions made by the first user 120 to make a medical diagnosis, monitor a medical condition, monitor treatment efficacy, look for specific symptoms, or the like.

Motion patterns 112 and user motions 114 may be organized in specific data structures or format to facilitate selective retrieval. Motion patterns 112 may also be referred to as a motion patterns library, motion patterns repository, motion signatures library, motion signatures repository, and the like. User motions 114 may also be referred to as a user motions library, user motions repository, and the like.

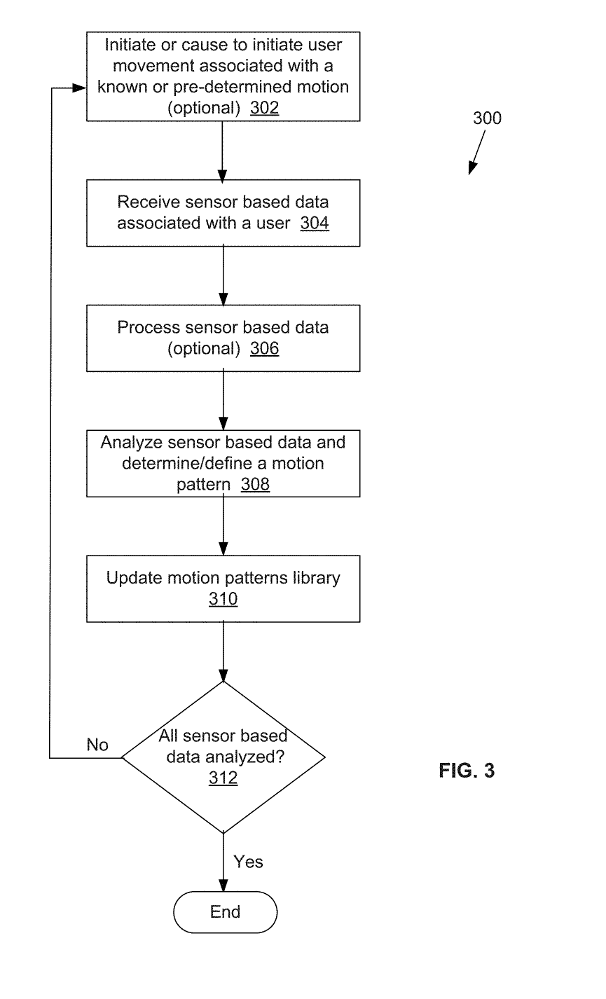

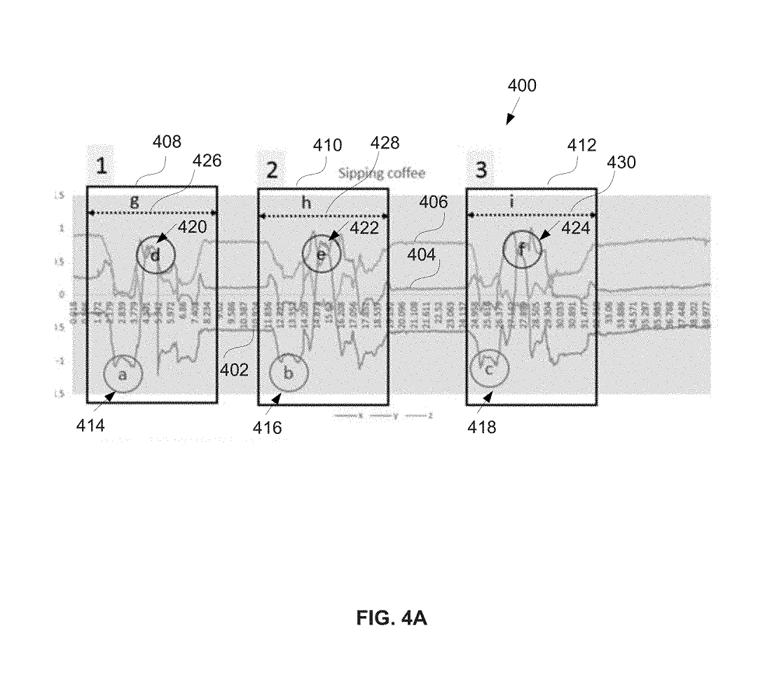

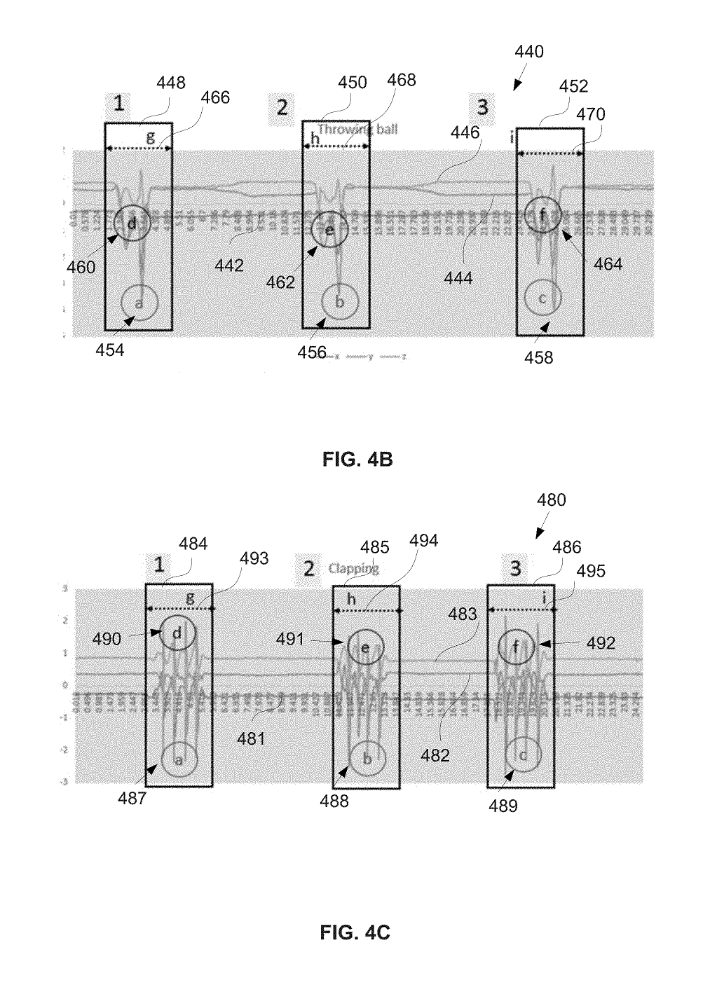

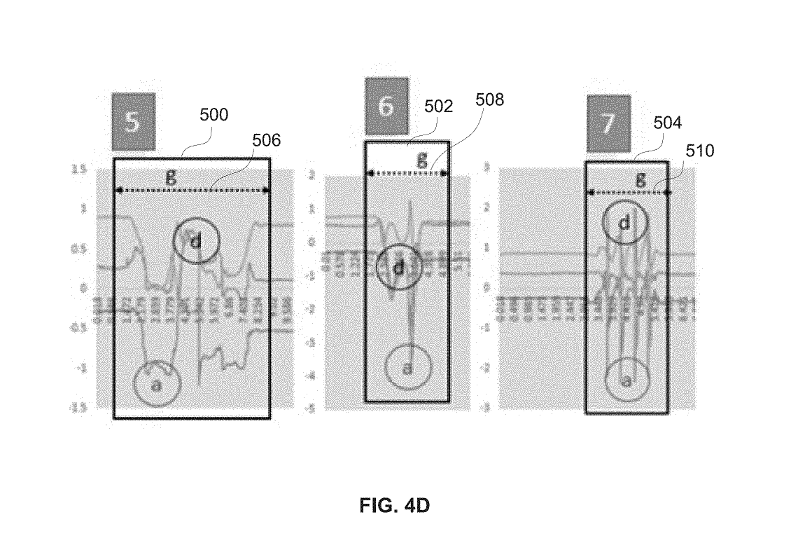

FIG. 3 depicts an example process 300 for training or building a library of motion patterns 112, according to some embodiments. FIGS. 4A-4D depict graphs illustrating example sensor based data associated with particular motions, according to some embodiments. FIG. 3 is discussed below in conjunction with FIGS. 4A-4D.

At block 302, the movement capture module 204 may initiate or cause to initiate a user (e.g., first user 120) to perform a movement for which the classification or type of motion is already known or pre-determined. For example, the movement capture module 204 may cause a device that the user is interfacing with to provide instructions for the user to perform a particular movement or action such as “take a sip of coffee,” “clap your hands together,” or “jump up and down three times.” In some embodiments, block 302 may be optional if the user is instructed by a person or some other mechanism outside of system 100 to perform the movement.

In response to the request, the user performs the requested movement, which in turn, is captured by one or more devices (e.g., devices 116) in contact with and/or in proximity to the user. The devices may then provide their sensor based data, which includes the movement information and possible associated information (e.g., user identifier, device identifier, date and time stamp, device location information, etc.), to the movement capture module 204 at block 304.

Next at block 306, the movement capture module 204 may process the received sensor based data, as necessary, suitable for use by the machine learning module 206. In some embodiments, the sensor based data received from the device(s) may benefit from filtering, normalization, format change, decryption, decompression, or other processing to transform the sensor based data for analysis and/or to compare with previously received sensor based data for the same known motion. In other embodiments, if the sensor based data has been pre-processed by the devices prior to transmission and/or they are already in a suitable form, block 306 may be omitted.

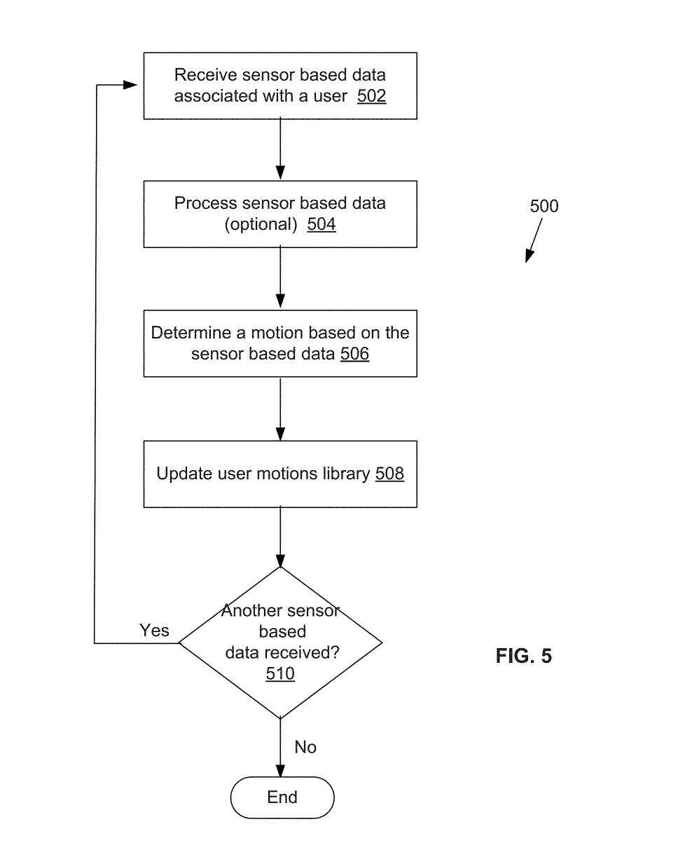

At block 308, the machine learning module 206 may analyze the sensor based data to determine or define a particular motion pattern or signature corresponding to the particular known motion. The particular motion pattern may specify the combination of sensor readings (and associated data such as time of day information) that are indicative of a particular movement by humans in general or a particular user, thereby providing a mechanism to automatically identify and classify movements captured in the future as a particular motion, as discussed in connection with FIG. 5. Machine learning module 206 may employ a variety of machine learning techniques, including but not limited to, statistical analysis, cluster analysis, image analysis, training sessions using known sensor based data for known motions, crowd sourcing, refinement over time, and the like. Data in addition to received sensor based data may also be used to define a motion pattern. For example, previous sensor based data may be used with the (current) sensor based data to define a motion pattern.

For example, user movement at a particular point in time may be captured by two devices: a first device (e.g., smartphone or wearable device) in contact with the user and including an accelerometer that captures accelerometer measurements of the movement, and a second device in proximity to the user (e.g., IoT device or webcam) including a camera that captures images of the user performing the movement. The accelerometer measurements and the images may comprise the sensor based data, which may be analyzed by the machine learning module 206 to “learn” what data points are recognizable as that movement.

In some embodiments, block 308 may not yield a motion pattern (e.g., if the sensor based data is corrupt or there is insufficient data to determine a motion pattern) or may merely yield a provisional motion pattern to be refined by additional sensor based data sets (repeating blocks 302-308 one or more times with subsequent sensor based data sets). This may be the case, for example, if a motion pattern associated with a new known motion is being defined.

The motion pattern (whether final, provisional, or other intermediate state) may then be stored as a motion pattern from among the plurality of motion patterns 112 by the motion database module 208 at block 310.

If there are additional sensor based data to be analyzed (e.g., machine learning is to continue to build the motion patterns library) (yes branch of block 312), then process 300 returns to block 302 for the next sensor based data. Otherwise (no branch of block 312), process 300 terminates.

In some embodiments, process 300 may be repeated one or more times for each respective known motion in order to define a motion pattern for each of the respective known motions. Process 300 may be performed more than once for a particular known motion, for example, periodically or over time to take into account movements made by new users and/or a greater number of users or to refine the motion pattern over time. Process 300 may be performed on a per user basis, in which a defined motion pattern may be associated with a particular user (as opposed to a group of users of a plurality of users in general).

FIG. 4A depicts a plot 400 illustrating example accelerometer measurements captured along each of three dimensions (e.g., x, y, and z Cartesian coordinates) over a time period during which a person performed a combination of movements that may be classified as “sipping coffee,” according to some embodiments. The vertical axis denotes the strength of the force of movements (e.g., g-force) in each of a first dimension 402 (e.g., x direction), a second dimension 404 (e.g., y direction), and a third dimension 406 (e.g., z direction). The horizontal axis denotes time.

Three sets of accelerometer measurements are shown—a first set of sensor based data 408, a second set of sensor based data 410, and a third set of sensor based data 412—associated with the person sipping coffee three times. In each of the first, second, and third sets of sensor based data 408, 410, 412, a portion of the data in the first dimension 402 has a distinct pattern that is similar to each other during approximately the same time during the sipping action. A first portion 414 (denoted as “a”), a second portion 416 (denoted as “b”), and a third portion 418 (denoted as “c”) share a similar pattern, for approximately the same time duration, and which occurs at approximately the same time that each sip is taken. Another portion of the data in the first dimension 402 also shows a distinct pattern that is similar to each other during approximately the same time across all three sips of coffee: a fourth portion 420 (denoted as “d”), a fifth portion 422 (denoted as “e”), and a third portion 424 (denoted as “f”). Likewise, time duration 426, 428, and 430 of respective sips of coffee are similar to each other.

These and other patterns associated with sipping coffee may be analyzed by the machine learning module 206 in block 308 of FIG. 3 to determine what x, y, and z forces measured by an accelerometer (and other possible information or sensor readings) are recognizable as the movement of sipping coffee (a classifiable motion). As may be appreciated, multiple data points with each sensor based data set and multiple data sets may be analyzed (e.g., more than three sets of sensor based data) to determine what data pattern reinforces each other (e.g., using comparative clustering techniques) because a person may not perform a movement exactly the same each time. One person may sip coffee differently than another person. Differences are expected and factored into during the training phase by the machine learning module 206.

When a large enough data set with a consistent pattern is determined, such data set may comprise a motion pattern. For example, the “sipping coffee” motion may be defined by a motion pattern comprising a combination of the first, second, and third sets of sensor based data 408, 410, 412. As another example, the motion pattern corresponding to the “sipping coffee” motion may comprise one of the first, second, and third sets of sensor based data 408, 410, 412 as a baseline set; one or more parameters specifying acceptable ranges, variations, and/or exceptions to the baseline set of sensor based data; and other possible limiters (e.g., time of day, user age, user location, etc.).

FIG. 4B depicts a plot 440 illustrating example accelerometer measurements captured along each of three dimensions (e.g., x, y, and z Cartesian coordinates) over a time period during which a person performed a combination of movements that may be classified as “throwing a ball,” according to some embodiments. The vertical axis denotes the strength of the force of movements (e.g., g-force) in each of a first dimension 442 (e.g., x direction), a second dimension 444 (e.g., y direction), and a third dimension 446 (e.g., z direction). The horizontal axis denotes time.

Three sets of accelerometer measurements are shown—a first set of sensor based data 448, a second set of sensor based data 450, and a third set of sensor based data 452—associated with the person throwing a ball three times. In each of the first, second, and third sets of sensor based data 448, 450, 452, a portion of the data in the first dimension 442 has a distinct pattern that is similar to each other during approximately the same time during the throwing action. A first portion 454 (denoted as “a”), a second portion 456 (denoted as “b”), and a third portion 458 (denoted as “c”) share a similar pattern, for approximately the same time duration, and which occurs at approximately the same time that each ball is thrown. Another portion of the data in the first dimension 442 also shows a distinct pattern that is similar to each other during approximately the same time across all three ball throwing action: a fourth portion 460 (denoted as “d”), a fifth portion 462 (denoted as “e”), and a third portion 464 (denoted as “f”). Likewise, time duration 466, 468, and 470 of respective ball throwing are similar to each other.

FIG. 4C depicts a plot 480 illustrating example accelerometer measurements captured along each of three dimensions (e.g., x, y, and z Cartesian coordinates) over a time period during which a person performed a combination of movements that may be classified as clapping hands together, according to some embodiments. The vertical axis denotes the strength of the force of movements (e.g., g-force) in each of a first dimension 481 (e.g., x direction), a second dimension 482 (e.g., y direction), and a third dimension 483 (e.g., z direction). The horizontal axis denotes time.

Three sets of accelerometer measurements are shown—a first set of sensor based data 484, a second set of sensor based data 485, and a third set of sensor based data 486—associated with the person clapping hands in three different bursts. In each of the first, second, and third sets of sensor based data 484, 485, 486, a portion of the data in the first dimension 481 has a distinct pattern that is similar to each other during approximately the same time during the clapping action. A first portion 487 (denoted as “a”), a second portion 488 (denoted as “b”), and a third portion 489 (denoted as “c”) share a similar pattern, for approximately the same time duration, and which occurs at approximately the same time that each set of clapping occurred. Another portion of the data in the first dimension 481 also shows a distinct pattern that is similar to each other during approximately the same time across all three clapping action: a fourth portion 490 (denoted as “d”), a fifth portion 491 (denoted as “e”), and a third portion 492 (denoted as “f”). Likewise, time duration 493, 494, and 495 of respective clapping bursts are similar to each other.

FIG. 4D depicts a plot contrasting example accelerometer measurements 500 for sipping coffee, accelerometer measurements 502 for throwing a ball, and accelerometer measurements 504 for clapping hands, according to some embodiments. Note how time duration 506, 508, and 510 for accelerometer measurements 502, 504, and 506 differ from each other, as well as the differences in amplitude and frequency of the measurements between the three different motions.

In this manner, a variety of motions may be defined in the database 110 in accordance with specific motion patterns determined from sensor based data. Although accelerometer measurements are discussed above in connection with FIGS. 4A-4D, it is contemplated that depending on the movement, other and/or additional sensors may be more appropriate to capture the movement information. For example, motions such as walking, running, or driving, may benefit from location detection sensors (e.g., GPS) in addition to accelerometers.

FIG. 5 depicts an example process 500 for automatically determining or classifying user movements, according to some embodiments. In some embodiments, process 500 may occur after one or more motion patterns are generated using process 300. In other embodiments, processes 500 and 300 may occur in parallel, in which process 300 continually or periodically applies machine learning techniques to received sensor based data to refine and update the motion patterns 112.

At block 502, the movement capture module 204 may receive sensor based data associated with a user (e.g., first user 120) generated by one or more devices (e.g., devices 116) in contact with and/or in proximity to the user. The sensor based data may comprise data points capturing one or more movements made by the user during a particular time period. The sensor based data may be similar to the sensor based data received in block 302 of FIG. 3.

In some embodiments, devices in contact with and/or in proximity to the user may automatically capture the user's movements throughout the day and night as the user goes about his or her day, and provide the captured movement information to the movement capture module 204 to initiate automatic determination and record of the user's motions for later use. The user need not initiate movement capture, a third party need not request movement capture, and the movement capture module 204 need not request sensor based data.

At block 504, the received sensor based data may be processed by the movement capture module 204 and/or motion determination module 210. The received sensor based data may be processed, on an as needed basis, into a form suitable for use in block 506. Similar to the discussion above for block 304 of FIG. 3, processing such as, but not limited to, filtering, normalizing, decrypting, decompressing, conversion, transformation, or other data processing may be performed. If no processing is necessary, block 504 may be omitted.

Next at block 506, the motion determination module 210 may automatically determine, classify, or recognize a motion corresponding to the sensor based data (or derivative thereof) from among the motions defined in accordance with the plurality of motion patterns 112. The sensor based data may be compared or analyzed against one or more records of the motion patterns 112 to determine a best match. Because each of the motion patterns 112 is associated with a particular motion (e.g., sipping coffee, throwing a ball, lifting a box, walking, bending over, etc.), finding the best matching motion pattern serves to identify the motion associated with the sensor based data.

With the user's current captured movement classified in block 506, the motion database module 208 updates the user motions 114, and in particular, the record(s) associated with the user, at block 508. In some embodiments, the user motions 114 may not include the received sensor based data because it is not needed once the motion corresponding to the received sensor based data has been determined. Instead, the record associated with the received sensor based data may comprise, for example: a user identifier (e.g., first user 120), a motion identifier of the motion determined in block 506, date/time stamp, and a location identifier (e.g., geographical coordinates, address, city, home or work, etc.).

The motion analysis module 106 waits for subsequent sets of sensor based data at block 510. If another set of sensor based data is received (yes branch of block 510), then process 500 may return to block 502. Otherwise, process 500 may end since no subsequent set of sensor based data is received (no branch of block 510). For example, if motion analysis module 106 is included in server 104, then sensor based data for a plurality of users may be received for classification. As another example, if motion analysis module 106 is included in a device 116, and there is no one in proximity to the device 116, then no movement may be sensed and hence, no sensor based data captured to be classified.

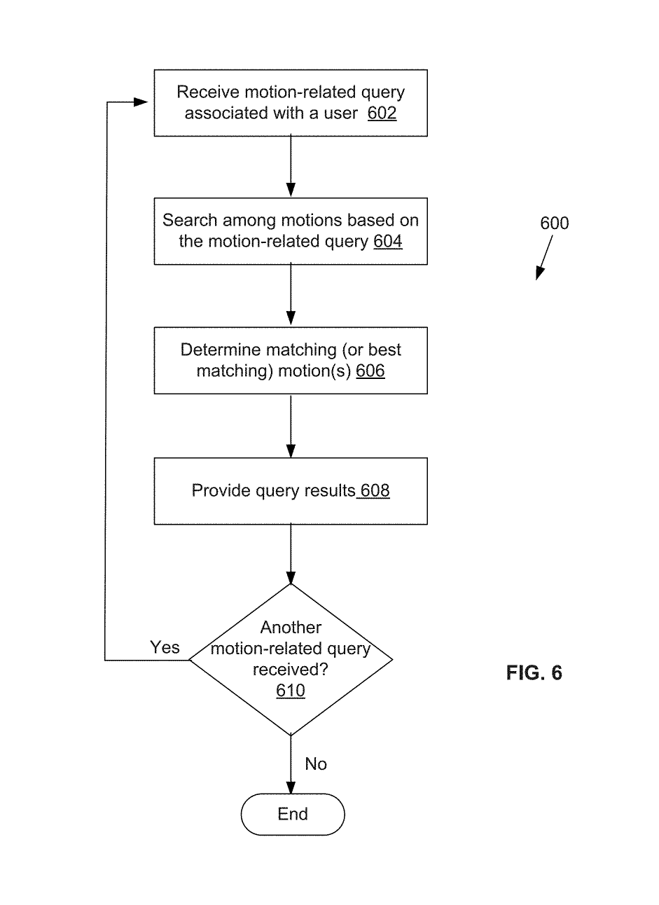

FIG. 6 depicts an example process 600 for using the user motions 114 to facilitate medical monitoring, medical diagnoses, object location recall, criminal investigations, and a variety of other purposes using classified motions about users, according to some embodiments. In some embodiments, process 600 may occur after one or more user motions 114 are generated using process 500.

At block 602, the query module 108 may receive a motion-related query associated with a user (e.g., first user 120). The motion-related query may be made by a user who is the same or different from the user about whom the query is directed. For example, the same user (e.g., first user 120) may request information about his or her past motions, or a different user (e.g., second user 122) may request information about the first user's 120 past motions. The query may also be composed in the same or different device from the device that captured the user's movement. For instance, the same device 116 (e.g., first user's 120 smartphone) may be used to both capture the first user's 120 movement and later request information about that movement. Or a different device 118 may be used to query motion(s) associated with the user that was captured via a device 116.

The motion-related query may comprise a single or compound query including, but not limited to, one or a combination of: a request for a particular type of motion; a request for motions during a particular time period; a request to identify a change in movement behavior over time; a request for times and/or locations when a particular motion occurred; a request to identify an increase or decrease in frequency of a particular motion; a request for preceding motions and contextually collected information such as calendar/schedule data, sounds, or visually (or other input-based) identified proximal objects (e.g., toaster, boxes, etc. that may relate to a movement associated with the user); and/or a request for particular metadata.

In some embodiments, queries may be automatically triggered and/or periodically composed. If, for example, the query results are sought by a third party (motions associated with the first user 120 are recalled by the second user 122), then the third party may periodically check the user's motions as a preventative measure or the third party may set parameters under which a query is automatically triggered.

In response to receiving the motion-related query, the query module 108 searches among records of user motions associated with the user, from among the user motions 114, at block 604. In some embodiments, the query module 108 may search motions identified in the user motions records associated with the user—as opposed to searching sensor based data or data points that require analysis or classification to motions. Because less processing or computational resources are needed to perform the search, faster reply time and less power consumption (relevant for mobile battery powered devices) may also be achieved. At block 606, the query module 108 determines the matching or best matching motion(s) from among the motions searched in block 604.

And at block 608, the query module 108 may provide the best or best matching motions as query results to the device that initiated the query. The query results may comprise one or more motions (e.g., “sipping coffee,” “lifting a box,” etc.), location information, date/time information, and/or other information associated with past user movements that were classified as particular motions and stored in the user motions 114.

If another motion-related query is received (yes branch of block 610), then process 600 may proceed to block 602. If no additional motion-related query is received (no branch of block 610), then process 600 may end.

In this manner, as a person goes about his/her business throughout a day, movements made by the person may be automatically captured, classified, and recorded for later use. Motions associated with the person may be discovered by comparing sensor based data from movement capture device(s) against a database of motions that correlates a form of the sensor based data (defined as motion patterns) with classified motions. The motions associated with the person may be stored compactly as motions rather than as all of the sensor data points that make up the motions. As a result, the person's motions may be easily stored, searched, and retrieved using lower powered (in terms of both computational power and electrical power) computing devices for a variety of uses.

Uses of the person's stored motions may include, but are not limited to, object retrieval, medical diagnosis, medical monitoring, better understanding of one's physical state, and the like. For example, if a person lost his wallet that was in his shirt front pocket, it is possible that the wallet fell out when he bent over. The person may compose a query requesting information about the times and locations when he bent over (e.g., querying for the motion “bending over” within the last 12 hours). The query module 106 may return a list of such times and locations. The person may then return to those locations to look for his wallet. As another example, a person may wake up with sore legs and may wonder as to the cause. He may query his past motions to determine what movement(s), if any, may be attributable to his current physical condition. The query results may include lifting heavy boxes, running up and down stairs, and/or other movements within the past 24 hours, and/or additionally indicate the amount of increase in such movements over time.

In still another example, certain medical conditions, such as obsessive compulsive disorder, may be associated with repetitive movements. Treatment of such medical conditions may be facilitated by looking for the occurrence of particular repetitive movements and/or the frequency of occurrence of particular repetitive movements. A relapse of the medical condition, for instance, may be identified by studying the occurrence of particular repetitive movements. As another example, a person may have an injury for which the cause is not obvious. Potentially damaging movements that may have occurred that caused the injury would be of interest. A medical personnel may query the user motions 114 for motions associated with the person during a particular time period to pinpoint one or more motions likely to be the cause of the injury. As another example, a person may stop or decrease certain movements that he/she has typically performed in the past. Because his/her movements are captured, classified, and stored in the user motions 114, any changes over time may be identified and an alert may be sent to the person and/or his doctor. The change may indicate an injury that the person is attempting to adjust to by changing their movement, or be a symptom of a new medical issue.

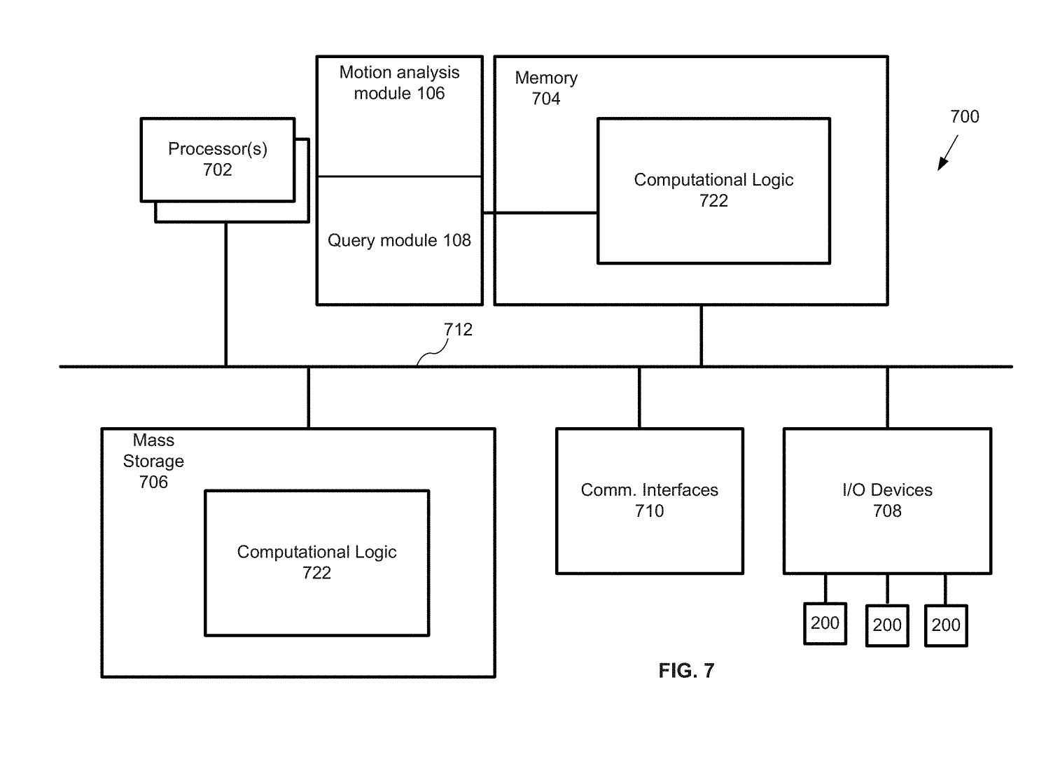

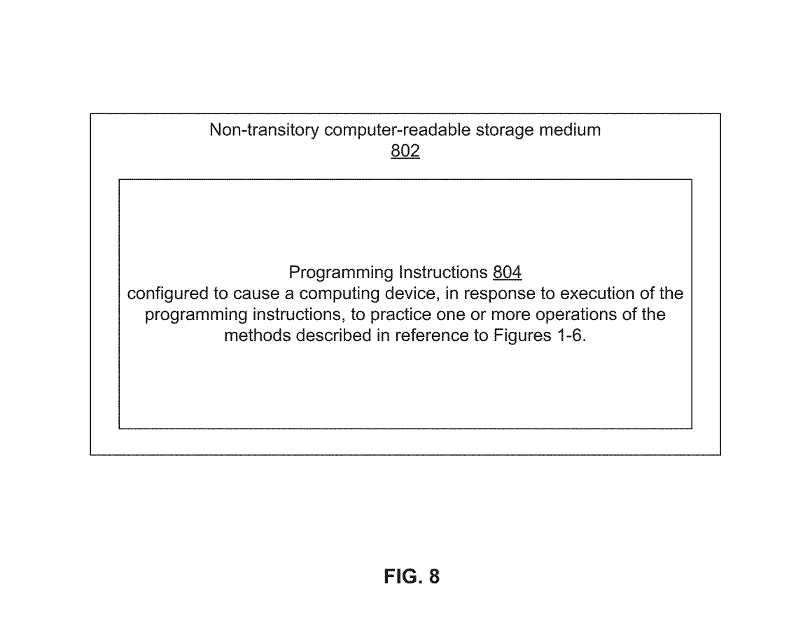

FIG. 7 illustrates an example computing device 700 suitable for use to practice aspects of the present disclosure, in accordance with various embodiments. In some embodiments, computing device 700 may comprise any of the server 104, database 110, devices 116, and/or devices 118. As shown, computing device 700 may include one or more processors or processor cores 702, and system memory 704. For the purpose of this application, including the claims, the terms “processor” and “processor cores” may be considered synonymous, unless the context clearly requires otherwise. The processor 702 may include any type of processors, such as a central processing unit (CPU), a microprocessor, and the like. The processor 702 may be implemented as an integrated circuit having multi-cores, e.g., a multi-core microprocessor. The computing device 700 may include mass storage devices 706 (such as diskette, hard drive, volatile memory (e.g., DRAM), compact disc read only memory (CD-ROM), digital versatile disk (DVD), flash memory, solid state memory, and so forth). In general, system memory 704 and/or mass storage devices 706 may be temporal and/or persistent storage of any type, including, but not limited to, volatile and non-volatile memory, optical, magnetic, and/or solid state mass storage, and so forth. Volatile memory may include, but not be limited to, static and/or dynamic random access memory. Non-volatile memory may include, but not be limited to, electrically erasable programmable read only memory, phase change memory, resistive memory, and so forth.

The computing device 700 may further include input/output (I/O) devices 708 (such as a display 202), keyboard, cursor control, remote control, gaming controller, image capture device, and so forth and communication interfaces 710 (such as network interface cards, modems, infrared receivers, radio receivers (e.g., Bluetooth)), and so forth. I/O devices 708 may further include and/or be coupled to sensors 200.

The communication interfaces 710 may include communication chips (not shown) that may be configured to operate the device 700 in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. The communication chips may also be configured to operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). The communication chips may be configured to operate in accordance with Code Division Multiple Access (CDMA), Time Division Multiple Access (TDMA), Digital Enhanced Cordless Telecommunications (DECT), Evolution-Data Optimized (EV-DO), derivatives thereof, as well as any other wireless protocols that are designated as 3G, 4G, 5G, and beyond. The communication interfaces 710 may operate in accordance with other wireless protocols in other embodiments.

The above-described computing device 700 elements may be coupled to each other via a system bus 712, which may represent one or more buses. In the case of multiple buses, they may be bridged by one or more bus bridges (not shown). Each of these elements may perform its conventional functions known in the art. In particular, system memory 704 and mass storage devices 706 may be employed to store a working copy and a permanent copy of the programming instructions implementing the operations associated with system 100, e.g., operations associated with providing motion analysis module 106 and query module 108 as described above, generally shown as computational logic 722. Computational logic 722 may be implemented by assembler instructions supported by processor(s) 702 or high-level languages that may be compiled into such instructions. The permanent copy of the programming instructions may be placed into mass storage devices 706 in the factory, or in the field, through, for example, a distribution medium (not shown), such as a compact disc (CD), or through communication interfaces 710 (from a distribution server (not shown)).

FIG. 8 illustrates an example non-transitory computer-readable storage media 802 having instructions configured to practice all or selected ones of the operations associated with the processes described above. As illustrated, non-transitory computer-readable storage medium 802 may include a number of programming instructions 804 (e.g., motion analysis module 106, query module 108). Programming instructions 804 may be configured to enable a device, e.g., computing device 700, in response to execution of the programming instructions, to perform one or more operations of the processes described in reference to FIGS. 1-6. In alternate embodiments, programming instructions 804 may be disposed on multiple non-transitory computer-readable storage media 802 instead. In still other embodiments, programming instructions 804 may be encoded in transitory computer-readable signals.

Referring again to FIG. 7, the number, capability, and/or capacity of the elements 708, 710, 712 may vary, depending on whether computing device 700 is used as a stationary computing device, such as a set-top box or desktop computer, or a mobile computing device, such as a tablet computing device, laptop computer, game console, or smartphone. Their constitutions are otherwise known, and accordingly will not be further described.

At least one of processors 702 may be packaged together with memory having computational logic 722 configured to practice aspects of embodiments described in reference to FIGS. 1-7. For example, computational logic 722 may be configured to include or access motion analysis module 106. In some embodiments, at least one of the processors 702 may be packaged together with memory having computational logic 722 configured to practice aspects of processes 300, 500, and/or 600 to form a System in Package (SiP) or a System on Chip (SoC).

In various implementations, the computing device 700 may comprise a laptop, a netbook, a notebook, an ultrabook, a smartphone, a tablet, an Internet of Things (IoT) device, a personal digital assistant (PDA), an ultra mobile PC, a mobile phone, a desktop computer, a server, a printer, a scanner, a monitor, a set-top box, an entertainment control unit, a digital camera, a portable music player, or a digital video recorder. In further implementations, the computing device 700 may be any other electronic device that processes data.

Although certain embodiments have been illustrated and described herein for purposes of description, a wide variety of alternate and/or equivalent embodiments or implementations calculated to achieve the same purposes may be substituted for the embodiments shown and described without departing from the scope of the present disclosure. This application is intended to cover any adaptations or variations of the embodiments discussed herein.

Examples of the devices, systems, and/or methods of various embodiments are provided below. An embodiment of the devices, systems, and/or methods may include any one or more, and any combination of, the examples described below.

Example 1 is an apparatus to facilitate motion-related diagnosis or monitoring, which may include one or more processors; one or more storage medium to store a plurality of motion patterns; a motion analysis module having first instructions to be executed by the one or more processors, to determine and store motion records for a user; wherein the motion analysis module is to receive, from a first device, first and second sensor data of the user at a first and a second time period, determine and store first and second motion records for the user based at least in part on the first and second sensor data, and the plurality of motion patterns; and a query module having second instructions to be executed by the one or more processors, to provide motion-related query match, wherein the query module is to receive, from a second device, a motion-related query associated with the user, search among the first and second motion records to determine a query match; and provide, to the second device, the query match including data associated with the first motion record or the second motion record.

Example 2 may include the subject matter of Example 1, and may further include that the first device is the same as the second device.

Example 3 may include the subject matter of any of Examples 1-2, and may further include that the plurality of motion patterns is defined based on known movements by a plurality of users.

Example 4 may include the subject matter of any of Examples 1-3, and may further include that the plurality of users excludes the first user.

Example 5 may include the subject matter of any of Examples 1-4, and may further include the motion analysis module having third instructions to be executed by the one or more processors, to analyze third sensor based data associated with the user and a particular known motion and fourth sensor based data associated with the user and the particular known motion, and to determine a particular sensor based data set associated with the particular known motion in accordance with the third and fourth sensor based data, wherein the particular sensor based data set defines a particular motion pattern from among the plurality of motion patterns, and the third sensor based data differs from the fourth sensor based data.

Example 6 may include the subject matter of any of Examples 1-5, and may further include the particular motion pattern is specific to the user, and the particular motion pattern comprises the first motion pattern.

Example 7 may include the subject matter of any of Examples 1-6, and may further include the particular motion pattern is associated with a particular motion for one or both of the user and another user.

Example 8 may include the subject matter of any of Examples 1-7, and may further include that the third instructions include one or more machine learning process instructions.

Example 9 may include the subject matter of any of Examples 1-8, and may further include the first sensor based data comprises raw sensor data from the first device or derived sensor data from the raw sensor data.

Example 10 may include the subject matter of any of Examples 1-9, and may further include the motion-related query comprises a request for a particular type of motion.

Example 11 may include the subject matter of any of Examples 1-10, and may further include the motion-related query comprises a request for motions associated with the user during a particular time period.

Example 12 may include the subject matter of any of Examples 1-11, and may further include the motion-related query comprises a query to identify a change in movement behavior over time.

Example 13 may include the subject matter of any of Examples 1-12, and may further include the first device is in physical contact with the user during the first time period.

Example 14 may include the subject matter of any of Examples 1-13, and may further include the first device is not in physical contact with the user during the first time period.

Example 15 may include the subject matter of any of Examples 1-14, and may further include that the first sensor based data comprises data from one or more of an accelerometer, a gyroscope, a barometric sensor, an ultrasonic sensor, a motion sensor, a location sensor, a global positioning system (GPS), an audio sensor, a visual sensor, a camera, an infrared sensor, radio detection and ranging (RADAR), a light radar (LIDAR), a tomographic sensor, or a vibration sensor.

Example 16 may include the subject matter of any of Examples 1-15, and may further include that the one or more storage medium includes the first and second motion records, each of the first and second motion records including a motion identifier, a date and time identifier, a location identifier, and a user identifier.

Example 17 is a computer-implemented method to facilitate motion-related diagnosis or monitoring, which may include receiving, from a first device, first sensor based data and second sensor based data associated with a user for a first and a second time period, respectively; determining whether the first and second sensor based data are respectively associated with a first motion pattern and a second motion pattern from among a plurality of motion patterns; when the first and second sensor based data are determined to be associated with the first and second motion patterns, identifying the first and second sensor based data to be first and second motions by the user; in response to receiving, from a second device, a motion-related query associated with the user, searching among the first motion and the second motion to determine a query match; and providing the query match comprising data associated with the first motion or the second motion.

Example 18 may include the subject matter of Example 17, and may further include that the first device is the same as the second device.

Example 19 may include the subject matter of any of Examples 17-18, and may further include receiving third sensor based data associated with the user, wherein the third sensor based data relates to a particular known motion; receiving fourth sensor based data associated with the user, wherein the fourth sensor based data relates to the particular known motion and the fourth sensor based data differ from the third sensor based data; and analyzing the third sensor based data and the fourth sensor based data to determine a particular sensor based data set associated with the particular known motion, the particular sensor based data set defining a particular motion pattern from among the plurality of motion patterns.

Example 20 may include the subject matter of any of Examples 17-19, and may further include the particular motion pattern is specific to the user, and the particular motion pattern comprises the first motion pattern.

Example 21 may include the subject matter of any of Examples 17-20, and may further include the particular motion pattern is associated with a particular motion for one or both of the user and another user.

Example 22 may include the subject matter of any of Examples 17-21, and may further include that analyzing the third sensor based data and the fourth sensor based data comprises using one or more machine learning processes.

Example 23 may include the subject matter of any of Examples 17-22, and may further include the first sensor based data comprises raw sensor data from the first device or derived sensor data from the raw sensor data.

Example 24 may include the subject matter of any of Examples 17-23, and may further include that receiving a motion-related query comprises receiving a motion-related query that requests for a particular type of motion.

Example 25 may include the subject matter of any of Examples 17-24, and may further include that receiving a motion-related query comprises receiving a motion-related query that queries about motions associated with the user during a particular time period.

Example 26 may include the subject matter of any of Examples 17-25, and may further include that receiving a motion-related query comprises receiving a motion-related query to identify a change in movement behavior over time.

Example 27 may include the subject matter of any of Examples 17-26, and may further include the first device is in physical contact with the user during the first time period.

Example 28 may include the subject matter of any of Examples 17-27, and may further include the first device is not in physical contact with the user during the first time period.

Example 29 may include the subject matter of any of Examples 17-28, and may further include that the first sensor based data comprises data from one or more of an accelerometer, a gyroscope, a barometric sensor, an ultrasonic sensor, a motion sensor, a location sensor, a global positioning system (GPS), an audio sensor, a visual sensor, a camera, an infrared sensor, radio detection and ranging (RADAR), a light radar (LIDAR), a tomographic sensor, or a vibration sensor.

Example 30 is one or more computer-readable storage medium comprising a plurality of instructions to cause an apparatus, in response to execution by one or more processors of the apparatus, which may include to receive, from a first device, first and second sensor based data associated with a user for a first and a second time period; determine whether the first and second sensor based data are associated with a first and a second motion pattern from among a plurality of motion patterns; when the first and second sensor based data are determined to be associated with the first and the second motion pattern, identify the first and second sensor based data to be first and second motions of the user; receive, from a second device, a motion-related query associated with the user; search among the first motion and the second motion to determine a query match; and provide, to the second device, the query match having data associated with the first motion or the second motion.

Example 31 may include the subject matter of Example 30, and may further include the first device is the same as the second device.

Example 32 may include the subject matter of any of Examples 30-31, and may further include that the plurality of instructions, in response to execution by the one or more processors of the apparatus, further cause to receive third sensor based data associated with the user, wherein the third sensor based data relates to a particular known motion; receive fourth sensor based data associated with the user, wherein the fourth sensor based data relates to the particular known motion and the fourth sensor based data differ from the third sensor based data; and analyze the third sensor based data and the fourth sensor based data to determine a particular sensor based data set associated with the particular known motion, the particular sensor based data set defining a particular motion pattern from among the plurality of motion patterns.

Example 33 may include the subject matter of any of Examples 30-32, and may further include the particular motion pattern is specific to the user, and the particular motion pattern comprises the first motion pattern.

Example 34 may include the subject matter of any of Examples 30-33, and may further include the particular motion pattern is associated with a particular motion for one or both of the user and another user.

Example 35 may include the subject matter of any of Examples 30-34, and may further include to analyze the third sensor based data and the fourth sensor based data comprises using one or more machine learning processes.

Example 36 may include the subject matter of any of Examples 30-35, and may further include the first sensor based data comprises raw sensor data from the first device or derived sensor data from the raw sensor data.

Example 37 may include the subject matter of any of Examples 30-36, and may further include to receive a motion-related query comprises receiving a motion-related query that requests for a particular type of motion.

Example 38 may include the subject matter of any of Examples 30-37, and may further include to receive a motion-related query comprises receiving a motion-related query that queries about motions associated with the user during a particular time period.

Example 39 may include the subject matter of any of Examples 30-38, and may further include to receive a motion-related query comprises receiving a motion-related query to identify a change in movement behavior over time.

Example 40 may include the subject matter of any of Examples 30-39, and may further include the first sensor based data comprises data from one or more of an accelerometer, a gyroscope, a barometric sensor, an ultrasonic sensor, a motion sensor, a location sensor, a global positioning system (GPS), an audio sensor, a visual sensor, a camera, an infrared sensor, radio detection and ranging (RADAR), a light radar (LIDAR), a tomographic sensor, or a vibration sensor.

Example 41 is an apparatus to facilitate motion-related diagnosis or monitoring, which may include means to receive, from a first device, first sensor based data and second sensor based data associated with a user for a first and a second time period, respectively; means for determining whether the first and second sensor based data are respectively associated with a first motion pattern and a second motion pattern from among a plurality of motion patterns; when the first and second sensor based data are determined to be associated with the first and second motion patterns, means for identifying the first and second sensor based data to be first and second motions by the user; in response to receiving, from a second device, a motion-related query associated with the user, means for searching among the first motion and the second motion to determine a query match; and means for providing the query match comprising data associated with the first motion or the second motion.

Example 42 may include the subject matter of Example 41, and may further include the first device is the same as the second device.

Example 43 may include the subject matter of any of Examples 41-42, and may further include means for receiving third sensor based data associated with the user, wherein the third sensor based data relates to a particular known motion; means for receiving fourth sensor based data associated with the user, wherein the fourth sensor based data relates to the particular known motion and the fourth sensor based data differ from the third sensor based data; and means for analyzing the third sensor based data and the fourth sensor based data to determine a particular sensor based data set associated with the particular known motion, the particular sensor based data set defining a particular motion pattern from among the plurality of motion patterns.

Example 44 may include the subject matter of any of Examples 41-43, and may further include the particular motion pattern is specific to the user, and the particular motion pattern comprises the first motion pattern.

Example 45 may include the subject matter of any of Examples 41-44, and may further include the particular motion pattern is associated with a particular motion for one or both of the user and another user.

Example 46 may include the subject matter of any of Examples 41-45, and may further include the first sensor based data comprises raw sensor data from the first device or derived sensor data from the raw sensor data.

Example 47 may include the subject matter of any of Examples 41-46, and may further include the means for receiving a motion-related query receives the motion-related query that requests a particular type of motion.

Example 48 may include the subject matter of any of Examples 41-47, and may further include the means for receiving a motion-related query receives the motion-related query that queries about motions associated with the user during a particular time period.

Example 49 may include the subject matter of any of Examples 41-48, and may further include the means for receiving a motion-related query receives the motion-related query to identify a change in movement behavior over time.

Example 50 may include the subject matter of any of Examples 41-49, and may further include the first device is in physical contact with the user during the first time period.

Example 51 may include the subject matter of any of Examples 41-50, and may further include the first device is not in physical contact with the user during the first time period.

Computer-readable media (including non-transitory computer-readable media), methods, apparatuses, systems, and devices for performing the above-described techniques are illustrative examples of embodiments disclosed herein. Additionally, other devices in the above-described interactions may be configured to perform various disclosed techniques.

Although certain embodiments have been illustrated and described herein for purposes of description, a wide variety of alternate and/or equivalent embodiments or implementations calculated to achieve the same purposes may be substituted for the embodiments shown and described without departing from the scope of the present disclosure. This application is intended to cover any adaptations or variations of the embodiments discussed herein. Therefore, it is manifestly intended that embodiments described herein be limited only by the claims.

We claim:

1. An apparatus to facilitate motion-related diagnosis or monitoring, comprising:
one or more processors;
one or more storage medium to store a plurality of motion patterns;
a motion analysis module having first instructions to be executed by the one or more processors, to determine and store motion records for a user; wherein the motion analysis module is to receive, from a first device, first and second sensor data of the user at a first and a second time period, determine and store first and second motion records for the user based at least in part on the first and second sensor data, and the plurality of motion patterns; and
a query module having second instructions to be executed by the one or more processors, to provide motion-related query match, wherein the query module is to receive, from a second device, a motion-related query associated with the user, search among the first and second motion records to determine a query match; and provide, to the second device, the query match including data associated with the first motion record or the second motion record.

2. The apparatus of claim 1, wherein the first device is the same as the second device.

3. The apparatus of claim 1, wherein the plurality of motion patterns is defined based on known movements by a plurality of users.

4. The apparatus of claim 3, wherein the plurality of users excludes the first user.

5. The apparatus of claim 1, wherein:
the motion analysis module having third instructions to be executed by the one or more processors, to analyze third sensor based data associated with the user and a particular known motion and fourth sensor based data associated with the user and the particular known motion, and to determine a particular sensor based data set associated with the particular known motion in accordance with the third and fourth sensor based data, wherein the particular sensor based data set defines a particular motion pattern from among the plurality of motion patterns, and the third sensor based data differs from the fourth sensor based data.

6. The apparatus of claim 5, wherein the particular motion pattern is specific to the user, and the particular motion pattern comprises the first motion pattern.

7. The apparatus of claim 5, wherein the particular motion pattern is associated with a particular motion for one or both of the user and another user.

8. The apparatus of claim 5, wherein the third instructions include one or more machine learning process instructions.

9. The apparatus of claim 1, wherein the first sensor based data comprises raw sensor data from the first device or derived sensor data from the raw sensor data.

10. The apparatus of claim 1, wherein the motion-related query comprises a request for a particular type of motion.

11. The apparatus of claim 1, wherein the motion-related query comprises a query to identify a change in movement behavior over time.

12. The apparatus of claim 1, wherein the first device is in physical contact with the user during the first time period.

13. The apparatus of claim 1, wherein the first device is not in physical contact with the user during the first time period.

14. The apparatus of claim 1, wherein the first sensor based data comprises data from one or more of an accelerometer, a gyroscope, a barometric sensor, an ultrasonic sensor, a motion sensor, a location sensor, a global positioning system (GPS), an audio sensor, a visual sensor, a camera, an infrared sensor, radio detection and ranging (RADAR), a light radar (LIDAR), a tomographic sensor, or a vibration sensor.

15. The apparatus of claim 1, wherein the one or more storage medium includes the first and second motion records, each of the first and second motion records including a motion identifier, a date and time identifier, a location identifier, and a user identifier.

16. A computer-implemented method to facilitate motion-related diagnosis or monitoring, the method comprising:
receiving, from a first device, first sensor based data and second sensor based data associated with a user for a first and a second time period, respectively;
determining whether the first and second sensor based data are respectively associated with a first motion pattern and a second motion pattern from among a plurality of motion patterns;
when the first and second sensor based data are determined to be associated with the first and second motion patterns, identifying the first and second sensor based data to be first and second motions by the user;
in response to receiving, from a second device, a motion-related query associated with the user, searching among the first motion and the second motion to determine a query match; and
providing the query match comprising data associated with the first motion or the second motion.

17. The method of claim 16, further comprising:
receiving third sensor based data associated with the user, wherein the third sensor based data relates to a particular known motion;
receiving fourth sensor based data associated with the user, wherein the fourth sensor based data relates to the particular known motion and the fourth sensor based data differ from the third sensor based data; and
analyzing the third sensor based data and the fourth sensor based data to determine a particular sensor based data set associated with the particular known motion, the particular sensor based data set defining a particular motion pattern from among the plurality of motion patterns.

18. The method of claim 16, wherein the first sensor based data comprises raw sensor data from the first device or derived sensor data from the raw sensor data.

19. The method of claim 16, wherein receiving a motion-related query comprises receiving a motion-related query that requests for a particular type of motion.

20. The method of claim 16, wherein receiving a motion-related query comprises receiving a motion-related query that queries about motions associated with the user during a particular time period.

21. The method of claim 16, wherein receiving a motion-related query comprises receiving a motion-related query to identify a change in movement behavior over time.

22. One or more computer-readable storage medium comprising a plurality of instructions to cause an apparatus, in response to execution by one or more processors of the apparatus, to:
receive, from a first device, first and second sensor based data associated with a user for a first and a second time period;
determine whether the first and second sensor based data are associated with a first and a second motion pattern from among a plurality of motion patterns;
when the first and second sensor based data are determined to be associated with the first and the second motion pattern, identify the first and second sensor based data to be first and second motions of the user;
receive, from a second device, a motion-related query associated with the user;
search among the first motion and the second motion to determine a query match;
provide, to the second device, the query match having data associated with the first motion or the second motion.

23. The computer-readable storage medium of claim 22, wherein the plurality of instructions, in response to execution by the one or more processors of the apparatus, further cause to:
receive third sensor based data associated with the user, wherein the third sensor based data relates to a particular known motion;
receive fourth sensor based data associated with the user, wherein the fourth sensor based data relates to the particular known motion and the fourth sensor based data differ from the third sensor based data; and
analyze the third sensor based data and the fourth sensor based data to determine a particular sensor based data set associated with the particular known motion, the particular sensor based data set defining a particular motion pattern from among the plurality of motion patterns.

24. The computer-readable storage medium of claim 22, wherein the particular motion pattern is specific to the user, and the particular motion pattern comprises the first motion pattern.

25. The computer-readable storage medium of claim 22, wherein the particular motion pattern is associated with a particular motion for one or both of the user and another user.
